(19) United States
(12) Patent Application Publication
(10) Pub. No.: US 2022/0071321 A1
(43) Pub. Date: Mar. 10, 2022

(54) VENTILATING VISOR

(71) Applicant: Gary Osmanoff, Marlboro, NJ (US)

(72) Inventor: Gary Osmanoff, Marlboro, NJ (US)

(21) Appl. No.: 17/014,612

(22) Filed: Sep. 8, 2020

(51) Int. Cl. A41D 13/11; A41D 13/002

(52) U.S. Cl. CPC A41D 13/1161; A41D 13/0025; A41D 13/1184

(57) ABSTRACT

A ventilating visor is adapted to be worn by a health care provider, who is also wearing a face mask with eyeglasses and/or a face shield. The ventilating visor comprises a visor band and a visor extension. The two ends of the visor band are connected by an adjustable visor strap which secures the ventilating visor on the wearer's forehead. The visor extension supports one or more fans, which direct ambient air downward into the mask, either directly or through tubing. The air pressure on the upper side of the mask diverts the wearer's respiration downward away from the wearer's eyeglasses and/or face shield.

### FIELD OF INVENTION

The present invention relates to the field of protective equipment, and more particularly to face coverings that incorporate ventilation.

### BACKGROUND OF THE INVENTION

Viral epidemics and pandemics have compelled health care providers to wear face masks, often combined with face shields, to protect themselves and their patients. A problem arises when respiration leakage from the face mask causes fogging of the wearer's eyeglasses and/or face shield. While various devices have been proposed for using fan-generated ventilation to prevent fogging of goggles, none have addressed this problem.

### SUMMARY OF THE INVENTION

The present invention is a ventilating visor which is adapted to be worn by a health care provider, such as a doctor or dentist, who is also wearing a face mask with eyeglasses and/or a face shield. The ventilating visor comprises a visor band and a visor extension. The visor band is configured to extend across the wearer's forehead, and the two ends of the visor band are connected by an adjustable visor strap which secures the ventilating visor on the wearer's head.

The visor extension is connected to the visor band either directly or by two pivot joints. The visor extension projects outward from the visor band above the wearer's face and above the face mask. The visor extension supports one or more fans, which are electrically connected to a battery supported by the visor extension or the visor strap. The fans direct ambient air downward into the mask, either directly or through tubing, so as to form a plenum of positive pressure in the upper side of the mask. The plenum of positive pressure in the upper side of the mask diverts the wearer's respiration downward, away from the wearer's eyeglasses and/or face shield so as to prevent fogging thereof. The ventilation provided by the fans also serves to dissipate respiration heat and prevent its build up within a face shield. Optionally, air filters are attached to the fan inlets in order to remove contaminants and pathogens from the ambient air.

The foregoing summarizes the general design features of the present invention. In the following sections, specific embodiments of the present invention will be described in some detail. These specific embodiments are intended to demonstrate the feasibility of implementing the present invention in accordance with the general design features discussed above. Therefore, the detailed descriptions of these embodiments are offered for illustrative and exemplary purposes only, and they are not intended to limit the scope either of the foregoing summary description or of the claims which follow.

### BRIEF DESCRIPTION OF THE DRAWINGS

FIG. 1 is a perspective view of the first embodiment of the present invention worn by a person who is also wearing eyeglasses, a face mask and a face shield;

FIG. 2 is a side perspective view of the first embodiment of the present invention with the integrated visor extension and face shield pivoted upward from the visor band;

FIG. 3 is a bottom perspective view of the first embodiment of the present invention;

FIG. 4 is a top perspective view of the first embodiment of the present invention; and

FIG. 5 is a side perspective view of the second embodiment of the present invention with the face shield pivoted upward from the visor band.

### DETAILED DESCRIPTION OF THE PREFERRED EMBODIMENT

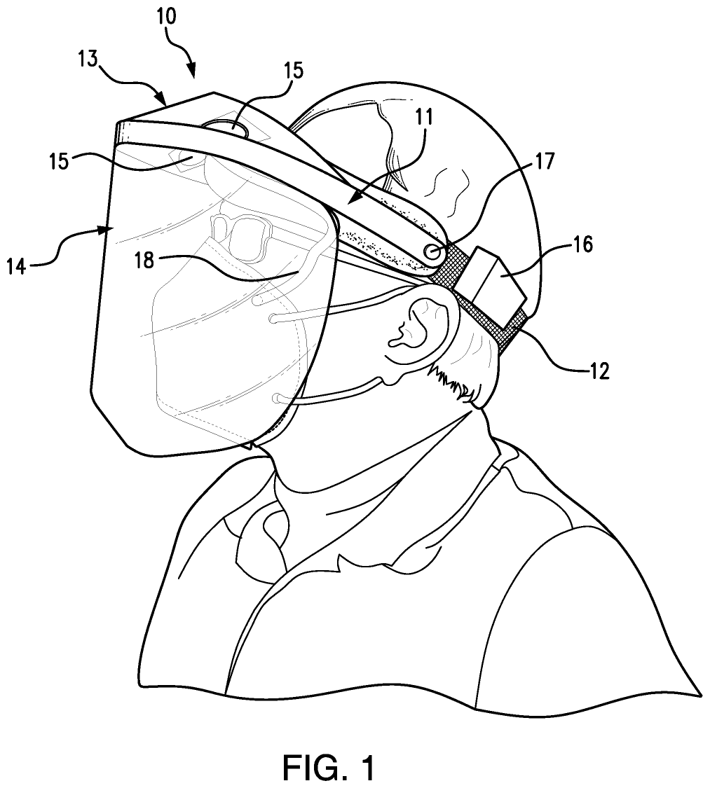

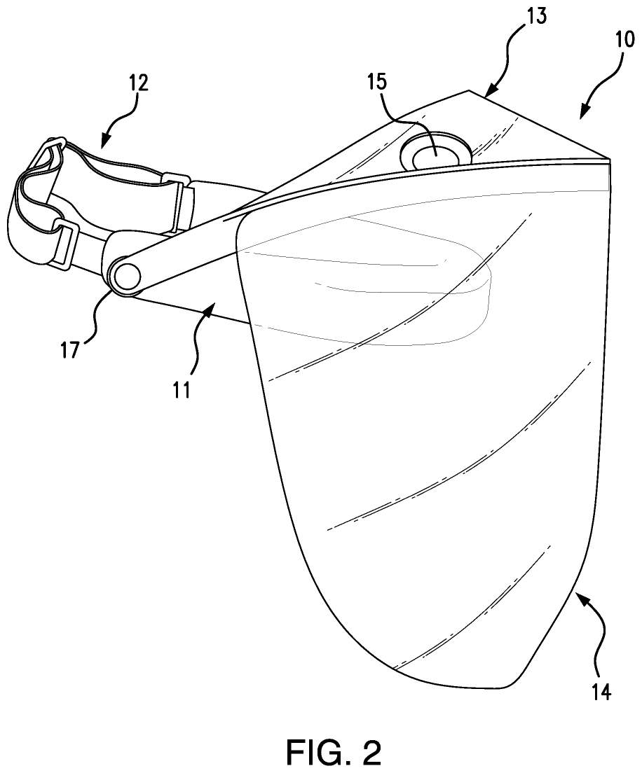

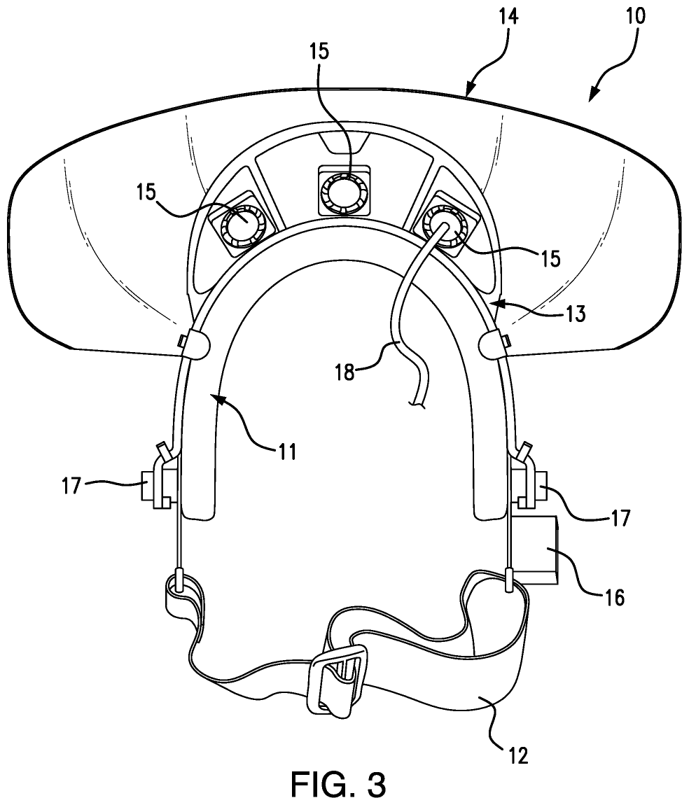

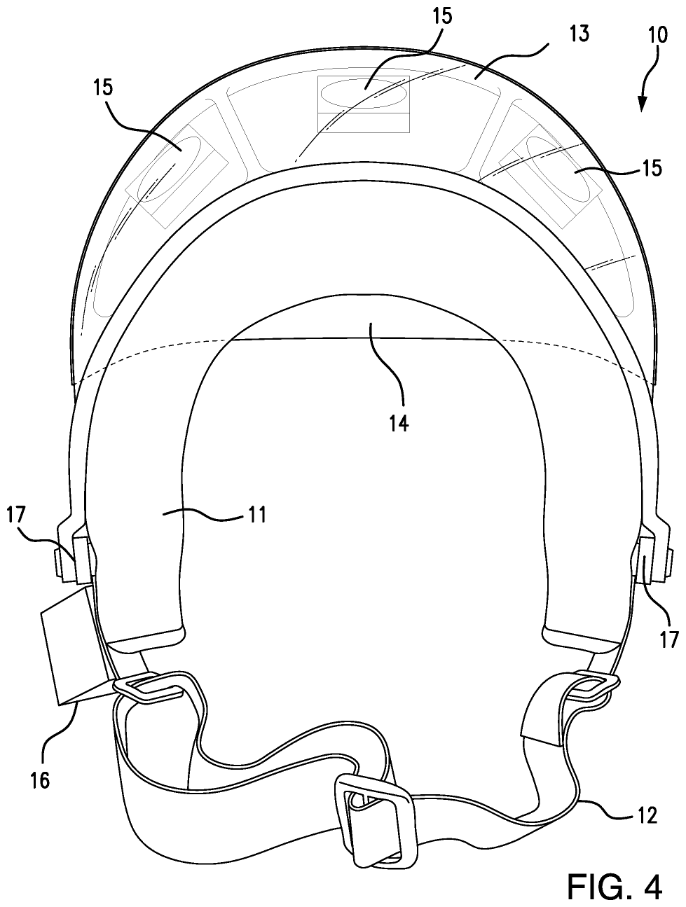

Referring to FIGS. 1-4, in the first exemplary embodiment of the present invention 10, the ventilating visor comprises the visor band 11, the visor strap 12 and the visor extension 13. The visor band 11 has generally semi-circular curvature and is made of a soft, flexible material, such as a plastic foam. The ends of the visor band are connected by the visor strap 12, which is an elastic material adjustable to secure the visor band to the wearer's forehead.

The visor extension 13 has a generally crescent shape with an inner curvature which matches the curvature of visor band 11, and it is made of a rigid, transparent plastic material. In this embodiment, the visor extension 13 has three embedded mini-fans 15, such as those used to cool computer CPUs. The outlet of one of the fans 15 is connected to tubing 18, which conveys the air flow into the wearer's mask, as best seen in FIG. 1. The fans 15 are electrically connected to a 9-volt battery 16, which is attached to the visor band 11.

In this first embodiment 10, the face shield 14 is attached to the outer edge of the visor extension 13. The visor extension 13 is pivotally attached to the visor band 11 through pivot connections 17 on either end of the visor band 11. This design enables the visor extension 13 and the face shield 14 to be raised and lowered together.

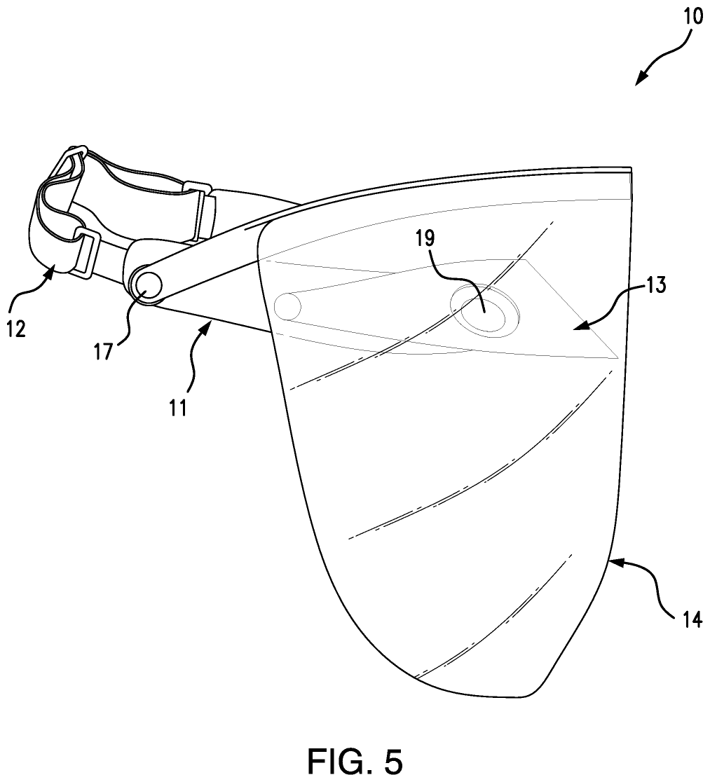

The second exemplary embodiment of the present invention 10 is illustrated in FIG. 5. Here, the visor extension 13 is directly attached to the visor band 11, and the face shield 14 is pivotally attached to the visor band 11 through the two pivot connections 17. In this design, the face shield 14 can be raised and lowered without moving the visor extension 13. In this second embodiment, the inlets of the fans 15 are covered by air filters 19, which remove contaminants and pathogens from the incoming air.

Although the preferred embodiments of the present invention have been disclosed for illustrative purposes, those skilled in the art will appreciate that many additions, modifications and substitutions are possible, without departing from the scope and spirit of the present invention as defined by the accompanying claims.

What is claimed is:

1. A ventilating visor adapted to be worn on a head of a wearer when wearing a face mask with eyeglasses and/or a face shield, wherein the face mask covers a lower portion of a face of the wearer, including a nose and a mouth of the wearer, the ventilating visor comprising:
a generally circularly curved flexible visor band, having two band ends and configured to extend across and engage the wearer's forehead;
an adjustable visor strap connecting the band ends and configured to secure the ventilating visor on the wearer's head;
a visor extension connected to the visor band and projecting outward from the visor band above the wearer's face and above the face mask;
one or more fans supported by the visor extension, wherein the fans direct ambient air downward toward the mask, so as to divert the wearer's respiration downward away from the wearer's eyeglasses and/or face shield and thereby prevent fogging thereof;
one or more batteries electrically connected to the fans and supported on the ventilating visor.

2. The ventilating visor according to claim 1, wherein the ventilating visor is adapted to be worn by the wearer when wearing the face mask with the eyeglasses and the face shield, and wherein the face shield is pivotally connected to the visor band, such that the face shield can be raised above the wearer's face and lowered over the wearer's face.

3. The ventilating visor according to claim 1, wherein the ventilating visor is adapted to be worn by the wearer when wearing the face mask with the eyeglasses and the face shield, and wherein the visor extension is pivotally connected to the visor band, and wherein the face shield is attached to the visor extension and extends downward from an outer edge of the visor extension, such that visor extension with the face shield attached can be raised above the wearer's face and lowered over the wearer's face.

4. The ventilating visor according to claim 1, wherein some or all of the fans direct ambient air downward into the mask through tubing.

5. The ventilating visor according to claim 2, wherein some or all of the fans direct ambient air downward into the mask through tubing.

6. The ventilating visor according to claim 3, wherein some or all of the fans direct ambient air downward into the mask through tubing.

7. The ventilating visor according to claim 1, wherein air filters are attached to some or all of the fans in order to remove contaminants from the ambient air.

8. The ventilating visor according to claim 2, wherein air filters are attached to some or all of the fans in order to remove contaminants from the ambient air.

9. The ventilating visor according to claim 3, wherein air filters are attached to some or all of the fans in order to remove contaminants from the ambient air.

10. The ventilating visor according to claim 4, wherein air filters are attached to some or all of the fans in order to remove contaminants from the ambient air.

11. The ventilating visor according to claim 5, wherein air filters are attached to some or all of the fans in order to remove contaminants from the ambient air.

12. The ventilating visor according to claim 6, wherein air filters are attached to some or all of the fans in order to remove contaminants from the ambient air.
